(19) United States
(12) Patent Application Publication
(10) Pub. No.: US 2024/0407712 A1
(43) Pub. Date: Dec. 12, 2024

(54) FOUR-COMPARTMENT CONTROLLER MODEL OF MUSCLE FATIGUE FOR ALL ACTIVITY TYPES

(71) Applicant: Texas Tech University System, Lubbock, TX (US)

(72) Inventors: James Yang, Lubbock, TX (US); Ritwik Rakshit, Lubbock, TX (US)

(73) Assignee: Texas Tech University System, Lubbock, TX (US)

(21) Appl. No.: 18/741,371

(22) Filed: Jun. 12, 2024

Related U.S. Application Data
(60) Provisional application No. 63/472,551, filed on Jun. 12, 2023.

(51) Int. Cl. A61B 5/00; A61B 5/11; G16H 20/30; G16H 50/20

(52) U.S. Cl. CPC A61B 5/4519; A61B 5/1114; A61B 5/7275; G16H 20/30; G16H 50/20

(57) ABSTRACT

Embodiments pertain to a computer-implemented method of predicting muscle fatigue in a subject by: (1) receiving data related to muscle fatigue in a muscle of the subject; (2) feeding the data into an algorithm to predict the muscle fatigue; and (3) outputting the predicted muscle fatigue. The algorithm my include: (a) an active compartment (M_A) model representing individual motor units (MUs) generating force at full capacity, (b) a resting compartment (M_R) model representing inactive MUs capable of rapid activation into force production, (c) a centrally fatigued compartment (M_FC) model representing fatigued MUs due to a central mechanism dominant at zero or near-zero joint velocities, with rapid recovery, and (d) a peripherally fatigued compartment (M_FP) model representing fatigued MUs due to a peripheral mechanism dominant at higher velocities, with slow recovery. Further embodiments pertain to a computing device for predicting muscle fatigue in a subject in accordance with such methods.

### CROSS-REFERENCE TO RELATED APPLICATIONS

This application claims priority to U.S. Provisional Patent Application No. 63/472,551, filed on Jun. 12, 2023. The entirety of the aforementioned application is incorporated herein by reference.

### STATEMENT REGARDING FEDERALLY SPONSORED RESEARCH

This invention was made with government support under Grant No. CBET 2014278 awarded by the National Science Foundation. The government has certain rights in this invention.

### BACKGROUND

A need exists for more effective methods and systems for predicting muscle fatigue. Numerous embodiments of the present disclosure aim to address the aforementioned need.

### SUMMARY

In some embodiments, the present disclosure pertains to a computer-implemented method of predicting muscle fatigue in a subject. In some embodiments, the methods of the present disclosure include: (1) receiving data related to muscle fatigue in a muscle of the subject; (2) feeding the data into an algorithm to predict the muscle fatigue; and (3) outputting the predicted muscle fatigue. In some embodiments, the methods of the present disclosure also include a step of recommending a treatment or minimization regimen for the muscle fatigue, such as modifications of tasks and/or modifications of workspaces. In some embodiments, the methods of the present disclosure also include a step of implementing a treatment or minimization regimen.

Further embodiments of the present disclosure pertain to a computing device for predicting muscle fatigue in a subject. In some embodiments, the computing device includes one or more computer readable storage mediums having a program code embodied therewith. In some embodiments, the program code includes programming instructions for: (1) receiving data related to muscle fatigue in a muscle of the subject; (2) feeding the data into an algorithm to predict the muscle fatigue; and (3) outputting the predicted muscle fatigue. In some embodiments, the program code also includes programming instructions for (4) recommending a treatment or minimization regimen for the muscle fatigue.

The methods and computing devices of the present disclosure can utilize various algorithms for predicting muscle fatigue. For instance, in some embodiments, the algorithm includes at least the following models for predicting muscle fatigue: (a) an active compartment (M_A) model representing individual motor units (MUs) generating force at full capacity, (b) a resting compartment (M_R) model representing inactive MUs capable of rapid activation into force production, (c) a centrally fatigued compartment (M_FC) model representing fatigued MUs due to a central mechanism dominant at zero or near-zero joint velocities, with rapid recovery, and (d) a peripherally fatigued compartment (M_FP) model representing fatigued MUs due to a peripheral mechanism dominant at higher velocities, with slow recovery.

### DESCRIPTION OF THE DRAWINGS

FIG. 1A illustrates a method of predicting muscle fatigue in a subject.

FIG. 1B illustrates a computing device for predicting muscle fatigue in a subject.

FIG. 2 provides a schematic diagram of a 4CCr model depicting the flow of motor units (MUs) between four states.

FIGS. 3A-3C provide data comparing the 3CCr and 4CCr models. FIG. 3A shows residual capacities for Task 1 as predicted by the 3CCr and 4CCr models. FIG. 3B shows relative contributions of central and peripheral mechanisms. FIG. 3C shows a pattern of modeled TL and V variation. Only the first 40 seconds are shown in FIG. 3C for clarity.

FIGS. 4A-4C provide additional data comparing the 3CCr and 4CCr models. FIG. 4A shows residual capacities for Task 2 as predicted by the 3CCr and 4CCr models. FIG. 4B shows relative contributions of central and peripheral mechanisms. FIG. 4C shows a pattern of modeled TL and V variation.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

According to the World Health Organization, approximately 1.71 billion people have musculoskeletal conditions worldwide. Musculoskeletal disorders (MSDs) are a leading cause of disability worldwide, impacting workforce employment, individual well-being, and quality of life. The three most common MSDs are trauma, back pain, and arthritis. Additionally, the rate of yearly MSDs outweighs that of circulatory and respiratory diseases.

Moreover, the cost of treating MSDs is greater than the treatment for many other health conditions. Depending on the area of the body, assessment of MSDs includes gait, arms, legs, and spine screenings; imaging of bones and joints; and blood tests.

Muscle fatigue is currently measured in clinical practice through qualitative rating scales, such as a 6-minute walk test, or through subjective questionnaires, such as the Multidimensional Fatigue Inventory (MFI), the Fatigue Severity Scale (FSS), and the Visual Analog Scale (VAS). Some methods utilize electromyography (EMG) to detect muscle fatigue.

However, there still remains a lack of an objective and standardized method for fatigue measurement in clinical practice. In research, a three-compartment controller fatigue model is used for providing estimates of muscle fatigue. However, this model is only suitable for isometric muscle contractions and excludes dynamic contractions.

In sum, a need exists for more quantitative methods and systems for predicting muscle fatigue. Numerous embodiments of the present disclosure aim to address the aforementioned need.

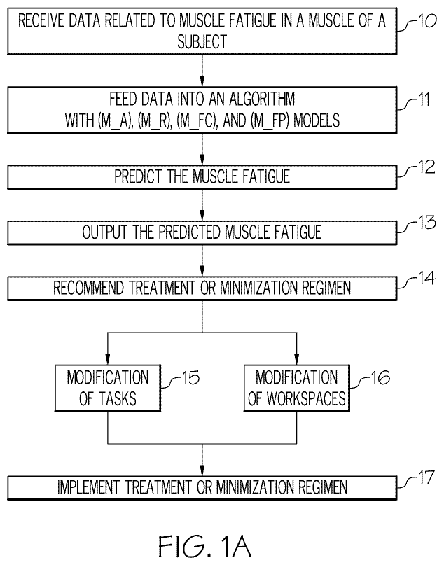

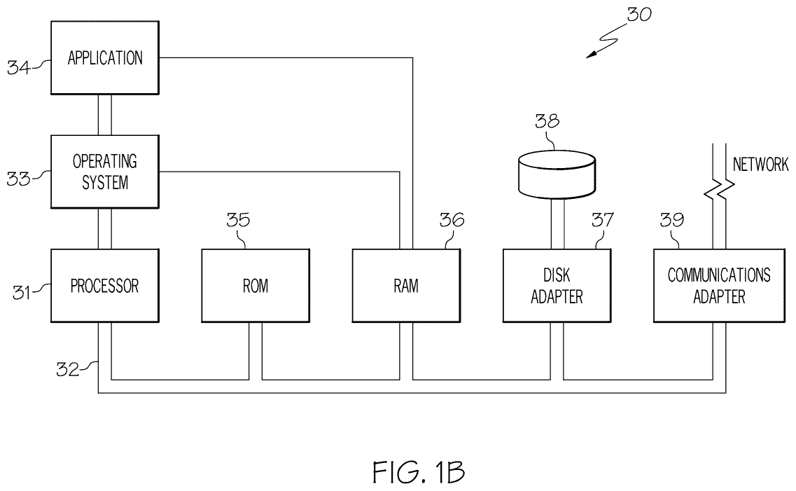

In some embodiments, the present disclosure pertains to a computer-implemented method of predicting muscle fatigue in a subject. In some embodiments illustrated in FIG. 1A, the methods of the present disclosure include: receiving data related to muscle fatigue in a muscle of the subject (step 10); feeding the data into an algorithm (step 11) to predict the muscle fatigue (step 12); and outputting the predicted muscle fatigue (step 13). In some embodiments, the methods of the present disclosure also include a step of recommending a treatment or minimization regimen for the muscle fatigue (step 14), such as modifications of tasks (step 15) and/or modifications of workspaces (step 16). In some embodiments, the methods of the present disclosure also include a step of implementing a treatment or minimization regimen (step 17).

Further embodiments of the present disclosure pertain to a computing device for predicting muscle fatigue in a subject. In some embodiments, the computing device includes one or more computer readable storage mediums having a program code embodied therewith. In some embodiments, the program code includes programming instructions for: (1) receiving data related to muscle fatigue in a muscle of the subject; (2) feeding the data into an algorithm to predict the muscle fatigue; and (3) outputting the predicted muscle fatigue. In some embodiments, the program code also includes programming instructions for (4) recommending a treatment or minimization regimen for the muscle fatigue.

As set forth in more detail herein, the methods and computing devices of the present disclosure can have numerous embodiments.

### Algorithms

The methods and computing devices of the present disclosure can utilize various algorithms for predicting muscle fatigue. For instance, in some embodiments, the algorithm includes at least the following models for predicting muscle fatigue: (a) an active compartment (M_A) model representing individual motor units (MUs) generating force at full capacity, (b) a resting compartment (M_R) model representing inactive MUs capable of rapid activation into force production, (c) a centrally fatigued compartment (M_FC) model representing fatigued MUs due to a central mechanism dominant at zero or near-zero joint velocities, with rapid recovery, and (d) a peripherally fatigued compartment (M_FP) model representing fatigued MUs due to a peripheral mechanism dominant at higher velocities, with slow recovery.

### M_A Models

The algorithms of the present disclosure can include various M_A models. For instance, in some embodiments, the generation of force at full capacity of the M_A model is represented by MVC (maximum voluntary contraction). In some embodiments, the M_A model is represented by the following formula:

$$\frac{{dM}_{A}}{dt} = - {F}_{P} {M}_{A} - {F}_{C} {M}_{A} + C ( t )$$

In some embodiments, MA represents the fraction of motor units activated, t represents time, F_{P} represents the peripheral fatigue coefficient, F_{C} represents the central fatigue coefficient, and C(t) represents the neural drive. In some embodiments, C(t) is represented by the following formula:

$$C ( t ) = L \times min ( TL - {M}_{A} , {M}_{R} )$$

In some embodiments, L represents a tracking factor, TL represents target load, M_{A} represents the fraction of motor units activated, and MR represents the fraction of motor units at rest. In some embodiments, F_{P} is represented by the following formula:

$${F}_{P} = {F}_{{P}_{0}} ( 1 - {e}^{- kV} )$$

In some embodiments, F_{P0} represents the baseline peripheral fatigue coefficient, e represents the base of the natural logarithm function, k represents the velocity coefficient, and V represents the joint angular velocity.

In some embodiments, F_{C} is represented by the following formula:

$${F}_{C} = {F}_{{C}_{0}} {e}^{- kV}$$

In some embodiments, F_{C0} represents the baseline central fatigue coefficient, e represents the base of the natural logarithm function, k represents the velocity coefficient, and V represents the joint angular velocity.

Unlike real motor units that are capable of modulating their force output over the full range, the individual motor units of the 4CCr model can either generate force at full capacity or generate no force at all. In some embodiments, modulation of total force output is achieved by varying the number of motor units activated into force production at any given time.

### M_R Models

The algorithms of the present disclosure can include various M_R models. For instance, in some embodiments, the rapid activation of inactive MUs into force production of the M_R model is represented by the rate of change of M_R. In some embodiments, the M_R model is represented by the following formula:

$$\frac{{dM}_{R}}{dt} = {R}_{P} {M}_{FP} + {R}_{C} {M}_{FC} - C ( t )$$

In some embodiments, M_{R} represents the fraction of motor units at rest, t represents time, R_{P} represents the peripheral recovery coefficient, M_{FP} represents the fraction of motor units affected by peripheral fatigue, R_{C} represents the central recovery coefficient, M_{FC} represents the fraction of motor units affected by central fatigue, and C(t) represents the neural drive. In some embodiments, C(t) is represented by the following formula:

$$C ( t ) = L \times min ( TL - {M}_{A} , {M}_{R} )$$

In some embodiments, L represents a tracking factor, TL represents target load, M_{A} represents the fraction of motor units activated, and M_{R} represents the fraction of motor units at rest. In some embodiments, R_{C} is represented by the following formula:

$${R}_{C} = \{ \begin{matrix}{R}_{{C}_{0}} if TL = 0 \\ {rR}_{{C}_{0}} if TL > 0\end{matrix}$$

In some embodiments, R_{C0} represents the baseline central recovery coefficient, r represents the augmented recovery coefficient, and TL represents target load.

### M_FC Model

The algorithms of the present disclosure can include various M_FC models. For instance, in some embodiments, the rapid recovery of the M_FC model is represented by a recovery time of less than about 1 hour. In some embodiments, the rapid recovery of the M_FC model is represented by a recovery time of less than about 60 seconds. In some embodiments, the zero or near-zero joint velocities of the M_FC model is represented by velocities ranging from 0 to a positive value (e.g., a small positive value) (degrees per second).

In some embodiments, the M_FC model is represented by the following formula:

$$\frac{{dM}_{FC}}{dt} = - {R}_{C} {M}_{FC} + {F}_{C} {M}_{A}$$

In some embodiments, M_{FC} represents the fraction of motor units centrally fatigued, t represents time, R_{C} represents the central fatigue coefficient, F_{C} represents the central fatigue coefficient, and M_{A} represents the fraction of motor units active.

In some embodiments, R_{C0} represents the baseline central recovery coefficient, r represents the augmented recovery coefficient, and TL represents target load.

### M_FP Model

The algorithms of the present disclosure can include various M_FP models. For instance, in some embodiments, the peripheral mechanism of the M_FP model is represented by the rate of change of M_{FP}. In some embodiments, the higher velocities of the M_FP model are represented by velocities above a positive value (e.g., a small positive value). In some embodiments, the slow recovery of the M_FP model is represented by a recovery time of more than about 1 hour.

In some embodiments, the M_FP model is represented by the following formula:

$$\frac{{dM}_{FP}}{dt} = - {R}_{P} {M}_{FP} + {F}_{P} {M}_{A}$$

In some embodiments, M_{FP} represents the fraction of motor units peripherally fatigued, t represents time, R_{P} represents the peripheral recovery coefficient, F_{P} represents the peripheral fatigue coefficient, and M_{A} represents the fraction of motor units active. In some embodiments, Rp is represented by the following formula:

$${R}_{P} = {R}_{{P}_{0}}$$

In some embodiments, R_{P0} represents the baseline peripheral recovery factor.

### Data Related to Muscle Fatigue

The methods of the present disclosure may feed various data related to muscle fatigue into an algorithm. Similarly, the computing devices of the present disclosure may include programming instructions for feeding various data related to muscle fatigue into an algorithm. For instance, in some embodiments, the data related to muscle fatigue includes joint velocity data. In some embodiments, the data related to muscle fatigue includes target load data. In some embodiments, the data related to muscle fatigue includes target load and joint velocity as functions of time for a muscle. In some embodiments, the data related to muscle fatigue includes central and peripheral fatigue and recovery coefficients.

### Predicting Muscle Fatigue

The algorithms of the present disclosure may be utilized to predict muscle fatigue in various manners. For instance, in some embodiments, the algorithm predicts muscle fatigue by predicting maximal force production capacity of the muscle over time. In some embodiments, the algorithm predicts muscle fatigue by predicting the instantaneous force production of the muscle as a fraction of its maximum force production capacity, based on the relative sizes and dynamics of the (M_A), (M_R), (M_FC), and (M_FP) models. In some embodiments, the algorithm predicts muscle fatigue by predicting the instantaneous force production of the muscle as a fraction of its maximum force production capacity using the M_A model.

The algorithms of the present disclosure may be utilized to predict various types of muscle fatigue. For instance, in some embodiments, the predicted muscle fatigue includes predicted muscle fatigue due to isometric muscle contraction. In some embodiments, the predicted muscle fatigue includes predicted muscle fatigue due to isokinetic muscle contraction. In some embodiments, the predicted muscle fatigue due to isokinetic muscle contraction represents muscle fatigue due to dynamic tasks, repetitive tasks, or combinations thereof.

In some embodiments, the predicted muscle fatigue includes predicted muscle fatigue due to isometric muscle contraction and isokinetic muscle contraction. In some embodiments, the predicted muscle fatigue includes prediction of changes in individual strength over the course of an activity given maximal voluntary contraction strength is measured.

A predicted muscle fatigue may be associated with various disorders. For instance, in some embodiments, the muscle fatigue is associated with a musculoskeletal disorder (MSD). In some embodiments, the musculoskeletal disorder is associated with trauma, back pain, arthritis, or combinations thereof.

### Muscles

The methods and computing devices of the present disclosure may be utilized to predict the muscle fatigue of various muscles. For instance, in some embodiments, the muscle includes, without limitation, a skeletal muscle, a smooth muscle, or combinations thereof.

### Outputting of Predicted Muscle Fatigue

The methods of the present disclosure may output a predicted muscle fatigue in various manners. Additionally, the computing devices of the present disclosure can include programming instructions to output a predicted muscle fatigue in various manners. For instance, in some embodiments, the outputting includes generating a report of the predicted muscle fatigue. In some embodiments, the outputting includes displaying the predicted muscle fatigue on a screen. In some embodiments, the screen includes a computer screen. In some embodiments, the screen includes a graphical user interface.

### Recommendation of a Treatment or Minimization Regimen

In some embodiments, the methods of the present disclosure include a step of recommending a treatment or minimization regimen for a predicted muscle fatigue. In some embodiments, the methods of the present disclosure include a step of implementing the treatment or minimization regimen. Similarly, the computing devices of the present disclosure may include programming instructions for recommending a treatment or minimization regimen for a predicted muscle fatigue.

In some embodiments, the treatment or minimization regimen includes modifications of tasks, modifications of workspaces, or combinations thereof. In some embodiments, the treatment or minimization regimen is aimed at treating or minimizing a musculoskeletal disorder. In some embodiments, the musculoskeletal disorder includes trauma, back pain, arthritis, or combinations thereof.

### Subjects

The methods and computing devices of the present disclosure may be utilized to predict muscle fatigue in various subjects. For instance, in some embodiments, the subject is a human being. In some embodiments, the subject is a non-human mammal. In some embodiments, the non-human mammal includes, without limitation, a horse, a rabbit, a mouse, a rat, a pig, a sheep, a cow, a dog, or a cat. In some embodiments, the non-human mammal is a domestic animal, such as a dog or a cat.

In some embodiments, the subject is suffering from a musculoskeletal disorder (e.g., trauma, back pain, and/or arthritis). In some embodiments, the subject is vulnerable to a musculoskeletal disorder.

### Computing Devices

The computing devices of the present disclosure can include various types of computer-readable storage mediums. For instance, in some embodiments, the computer-readable storage medium can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, or combinations thereof.

A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, or combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions for computing devices can be downloaded to respective computing/processing devices from a computer-readable storage medium or to an external computer or external storage device via a network, such as the Internet, a local area network (LAN), a wide area network (WAN) and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages.

In some embodiments, the computer-readable program instructions may execute entirely on the user's computer, partly on the user's computer, as a stand-alone software package, partly on the user's computer and partly on a remote computer or entirely on the remote computer or server. In the latter scenario, the remote computer may be connected in some embodiments to the user's computer through any type of network, including a LAN or a WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure for predicting muscle fatigue as discussed herein may be implemented using a computing device illustrated in FIG. 1B. Referring now to FIG. 1B, FIG. 1B illustrates an embodiment of the present disclosure of the hardware configuration of a computing device 30 which is representative of a hardware environment for practicing various embodiments of the present disclosure.

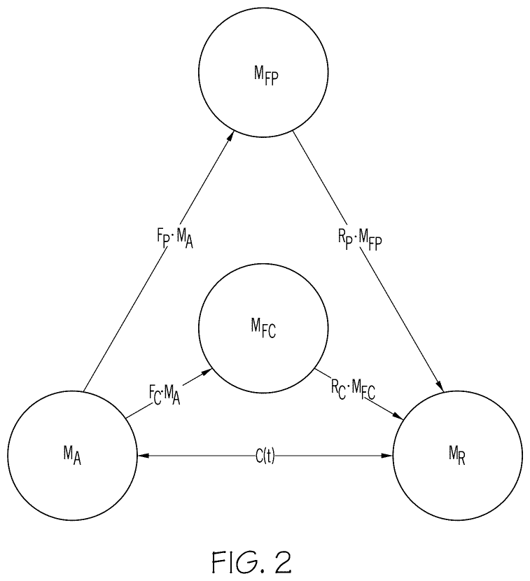

Computing device 30 has a processor 31 connected to various other components by computing device bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for predicting muscle fatigue as discussed in the present disclosure, such as in connection with FIGS. 2, 3A-3C, and 4A-4C.

Referring again to FIG. 1B, read-only memory (“ROM”) 35 is connected to computing device bus 32 and includes a basic input/output computing device (“BIOS”) that controls certain basic functions of computing device 30. Random access memory (“RAM”) 36 and disk adapter 37 are also connected to computing device bus 32. It should be noted that software components including operating system 33 and application 34 may be loaded into RAM 36, which may be computing device's 30 main memory for execution. Disk adapter 37 may be an integrated drive electronics (“IDE”) adapter that communicates with a disk unit 38 (e.g., a disk drive). It is noted that the program for predicting muscle fatigue, as discussed in the present disclosure, such as in connection with FIGS. 2, 3A-3C, and 4A-4C may reside in disk unit 38 or in application 34.

Computing device 30 may further include a communications adapter 39 connected to computing device bus 32. Communications adapter 39 interconnects computing device bus 32 with an outside network (e.g., wide area network) to communicate with other devices.

Aspects of the present invention are described herein with reference to flowchart illustrations and/or block diagrams of methods, apparatus (systems), and systems according to embodiments of the invention. It will be understood that each block of the flowchart illustrations and/or block diagrams and combinations of blocks in the flowchart illustrations and/or block diagrams can be implemented by computer-readable program instructions.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of computing devices, methods, and computing devices according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based computing devices that perform the specified functions or acts or carry out combinations of special purpose hardware and computer instructions.

### Applications and Advantages

The methods and computing devices of the present disclosure provide numerous advantages. For instance, in some embodiments, the computing devices and methods of the present disclosure allow for enhanced accuracy for downstream predictions and modeling of extended tasks or missions. Moreover, in some embodiments, the computing devices and methods of the present disclosure can be utilized to predict fatigue for isometric muscle contraction-related tasks, isokinetic muscle contraction-related tasks, dynamic muscle contraction-related tasks, or any combination thereof. As such, the methods and computing devices of the present disclosure can be utilized to predict muscle fatigue caused by different types of activities.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. A Four-Compartment Model to Predict Central and Peripheral Contributions of Fatigue

Compartment based models of fatigue have been particularly successful in accurately modeling isometric tasks or actions. Dynamic actions, which make up the majority of everyday movements, are governed by different central and peripheral processes, and must therefore be modeled in a manner accounting for the differences in the responsible mechanisms. In this Example, Applicant outlines the general framework of a four-compartment model that allows the modeling of central and peripheral fatigue separately and estimates strength decline for tasks involving either or both.

### Example 1.1. Background

Joint velocity is used as an indicator of the degree of contribution of either mechanism. Joint velocity maintains the predictions of the extensively validated three compartment controller model for isometric tasks but provides divergent predictions for isokinetic activities (increasing fatigue with increasing velocity) in line with experimentally observed trends.

Localized muscular fatigue (LMF) is an exercise-induced reduction in the ability of a muscle to generate force or power and is an important consideration in obtaining accurate estimates of strength. While LMF is complex in its origins, all causative mechanisms can be classified as either central or peripheral. Central mechanisms originate in the central nervous system (CNS) and result in the impaired voluntary activation of motor units (MU). Peripheral mechanisms, on the other hand, involve any and all effects distal to the neuromuscular joint, including but not limited to muscle action potential propagation, excitation-contraction coupling (ECC), and chemical changes within the contractile elements in muscle.

Numerous studies have investigated the relation between muscle shortening velocity, strength decline, and the mechanisms responsible with varied results. Increasing peak torque loss with increasing velocity has previously been observed, although a single report exists of greater decline at a lower shortening velocity. The apparently anomalous result may be attributed to the difference in the protocol parameters used as the number of contractions and duty cycle and cycle time differed widely between the studies. The weight of evidence supports the notion that increasing velocities lead to increased fatigue, although a definitive assertion has yet to be made regarding the exact manner in which this occurs.

For instance, a greater change in the contractile properties of muscle after concentric contraction was observed than after isometric contractions, indicating a tendency for concentric tasks to be dominated by peripheral fatigue. Additionally, it has been noticed that isometric tasks were predominantly influenced by central mechanisms without a decrease in voluntary activation.

The difference in duty cycle and cycle time between the protocols may have been responsible for the different observations. Studies have also observed a decrease in neural drive for 60°/s isokinetic contractions compared to those at 120°/s for the knee extensors, likely attributed to an inhibition of the lb afferents originating from the Golgi tendon organs.

Due to the multiple differences in protocol between each of the experimental studies with regards to joints tested and task parameters, it is challenging to derive a precise relationship between the relative contributions of central vs. peripheral mechanisms at any given velocity without further standardized experiments. The majority of the studies do indicate, however, that one or more central mechanisms are likely dominant at lower shortening velocities and isometric actions, and that as the muscle shortening velocity increases, so does the contribution of peripheral mechanisms.

A study has reported a rapid restitution of voluntary force after brief, high-intensity exercise attributable to a recovery of central mechanisms within 2 minutes and certain peripheral aspects such as excitation-contraction coupling and muscle reperfusion within 5 minutes. Complete recovery, however, was found to take hours due to the prolonged peripheral impairment in intracellular Ca²⁺ release or sensitivity.

Mathematical models of LMF have sought to predict the decline in peak force/torque under various task conditions using many approaches. The most occupationally relevant models are computationally simple, require as input only data that is readily available, and can realistically depict the processes of fatigue and recovery under specified task conditions.

Compartment theory has often been used to model transport phenomena in chemical reactions and biological processes, most notably in epidemiology. They depict material flow between the components of a system, and the rules governing the flow are usually represented deterministically by ordinary differential equations. Stochastic relations may be used to analyze the probabilistic response of the system but tend to make computations more difficult. The compartments in the model do not always correspond to physical or physiological analogues, but can help determine the parameters that best represent the phenomenon being described. Compartment models have proved to be a particularly useful technique for LMF using only the time varying target load (TL) as input. In an occupational setting, the simplicity of this method has the unique advantage of being able to generate predictions without using any biological measurements such as surface electromyography (sEMG), oxygen consumption, or carbon dioxide production.

A study first used compartment theory to model LMF by dividing muscles into three states: resting, active, and fatigued. In their approach, resting/recovered motor units could be recruited into the active state, which could then move into the fatigued state. Fatigued motor units were allowed to be directly activated when needed, but never allowed to return to the resting state. Complete recovery was precluded by this approach but was addressed in a three-compartment controller (3CC) model. The 3CC model rearranged the flow between the same 3 compartments so all motor units could return to the recovered state once the activation drive was switched off.

Critically, it was also proposed that dynamic loads could be modeled by expressing the instantaneous desired torque as a fraction of the peak achievable torque at a given joint angle and joint velocity. This would allow the model input (target load (TL)) to remain a fractional value but would be calculated from empirically determined 3-dimensional peak torque-velocity-angle (TVA) surfaces using torque-velocity-angle data triplets from the activity. To Applicant's knowledge, this capability has not so far been validated.

In an update to the 3CC model, recovery during rest periods was enhanced in the 3CCr model to improve prediction accuracy for intermittent isometric tasks. The 3CCr model based on compartment theory has been validated against an extensive dataset of various isometric tasks and provides reasonable predictions for strength decline in isometric tasks, both sustained and intermittent. However, the 3CCr model cannot distinguish between isometric and isokinetic tasks, and predicts identical torque declines for both as long as TL is equal between the two. The 3CCr model also does not distinguish between the responsible fatigue/recovery mechanisms, which avoids needless complexity so long as it only deals with one activity type. However, if it is to be applied to tasks with varying contraction velocities (such as through the use of TVA surfaces), the etiology of fatigue under each condition must be carefully considered and accounted for.

In the remainder of this Example, Applicant describes the construction and predictions of a modified model based on the original 3CCr which addresses the previously mentioned challenges.

### Example 1.2. Methods

In the 3CCr model, active MUs would be allowed to pass into a single fatigued state. In Applicant's four-compartment controller model of fatigue with enhanced recovery (4CCr), Applicant divides the fatigued compartment of the 3CCr model into two: peripherally fatigued, and centrally fatigued. Accordingly, the governing equations for the four compartments depicted in FIG. 2 are as follows:

$$\frac{{dM}_{A}}{dt} = - {F}_{P} {M}_{A} - {F}_{C} {M}_{A} + C ( t ) \qquad ( 1 )$$

$$\frac{{dM}_{R}}{dt} = {R}_{P} {M}_{FP} + {R}_{C} {M}_{FC} - C ( t ) \qquad ( 2 )$$

$$\frac{{dM}_{FP}}{dt} = - {R}_{P} {M}_{FP} + {F}_{P} {M}_{A} \qquad ( 3 )$$

$$\frac{{dM}_{FC}}{dt} = - {R}_{C} {M}_{FC} + {F}_{C} {M}_{A} \qquad ( 4 )$$

In the aforementioned equations, M_{A}, M_{R}, M_{FC}, and M_{FP} are the sizes of the active, resting, centrally fatigued, and peripherally fatigued compartments, respectively, expressed as the fraction of all MUs occupying that particular state at a given time. C(t) retains its definition from the 3CCr model as a bidirectional neural drive that serves to transition MUs between the active and resting states depending on the instantaneous requirement of the task:

$$C ( t ) = L \times min ( T L - {M}_{A} , {M}_{R} ) \qquad ( 5 )$$

In equation 5, L is a tracking factor that ensures the developed force quickly and closely tracks TL. Extremely small values can cause poor tracking, but any value greater than ˜10 is sufficient to ensure that the model responds to changes in TL quickly.

Each of the new fatigued compartments has its unique set of associated F and R values corresponding to the expected relative rates of fatigue and recovery due to peripheral and central mechanisms, respectively. The recovery rate constant for peripheral fatigue R_{P} is assumed to have a constant value R_{P0} throughout:

$${R}_{P} = {R}_{{P}_{0}} \qquad ( 6 )$$

In accordance with the 3CCr model, R_{C} assumes ones of two discrete values depending on whether TL is non-zero or not:

$${R}_{C} = \{ \begin{matrix}{R}_{{C}_{0}} if TL = 0 \\ {rR}_{{C}_{0}} if TL > 0\end{matrix} \qquad ( 7 )$$

In Equation 7, R_{C0} is the baseline recovery rate coefficient for central fatigue, and r is the augmented recovery rate coefficient for rest. F_{P} and F_{C} vary continuously with velocity V. During an isometric task (V=0), F_{C}=F_{C0} and F_{P}=0. With increasing velocity F_{P} increases while F_{C} decreases according to Equations 7-8:

$${F}_{P} = {F}_{{P}_{0}} ( 1 - {e}^{- kV} ) \qquad ( 8 )$$

$${F}_{C} = {F}_{{C}_{0}} {e}^{- kV} \qquad ( 9 )$$

In equations 8 and 9, k is the velocity coefficient having the units s/°. In the trivial case when velocity is 0, the 4CCr equations collapse into those describing 3CCr and the resulting outputs are identical. 4CCr, therefore, generates the same predictions for isometric tasks (whether sustained or intermittent) as its predecessor. Both models are constructed in MATLAB/SIMULINK (The Math Works Inc., Natick, Massachusetts, USA) to compare their predictions of residual capacity, defined as the sum of the active and resting compartment sizes, for two non-trivial cases/tasks. The parameter values used for each model are described in Table 1.

TABLE 1
Generic parameter values used for constructing
the 3CCr and 4CCr models.

| | |
|---|---|
| 3CCr | 4CCr |

| Parameter | Value | Parameter | Value |
|---|---|---|---|
| F | 0.015 | F_{C0} | 0.015 |
| R | 0.00149 | R_{C0} | 0.00149 |
| | | F_{P0} | 0.015 |
| | | R_{P0} | 0.000015 |
| r | 15 | R | 15 |
| | | K | 0.015 s/° |
| L | 40 | L | 40 |

Task 1 involves continuous maximal effort isokinetic knee flexion and extension at 90°/s for a period of 200 s. The model parameters are, by design, muscle group specific, and in this case are chosen to represent the knee extensors. Since both flexion and extension occur at the same velocity, the duty cycle is 0.5. The cycle time is taken as 1.67 s for a range of motion (ROM) of 75°. With the activity modeled as maximal effort, TL is taken to be 1 during the extension phase (for the knee extensors) and 0 during the flexion phase.

Task 2 is more complex, and involves 5 bouts of maximal effort intermittent isometric activity lasting 90 s each interspersed by 4 bouts of maximal effort isokinetic knee flexion and extension at 150°/s lasting 60 s each, for a total activity time of 690 s. Within each identical isometric bout, isometric knee extension is performed 6 times, each lasting 3 s followed by a 12-s period of rest. Isokinetic knee flexion/extension is performed within a range of motion (ROM) of 75°. As in Task 1, TL is set to 1 during extension and to 0 during flexion or rest.

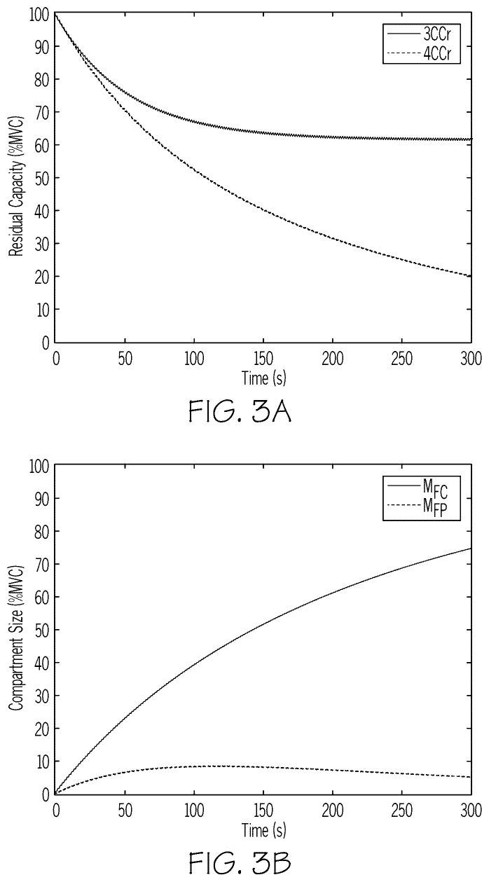

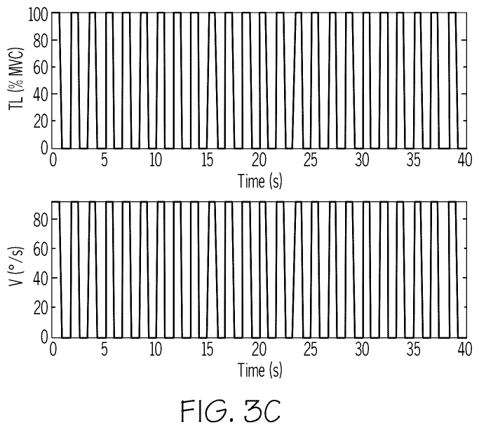

The residual capacities predicted by both models for Task 1 are widely divergent, as seen in FIG. 3A. Considering the predictions of the 4CCr model regarding the contributing mechanisms of fatigue as seen in FIG. 3B, central fatigue increases from zero, peaks at less than 10% MVC, and then declines slowly. Peripheral fatigue increases monotonically as the activity progresses. FIG. 3C depicts the variation of target load (top) and joint angular velocity (bottom) for the Task 1.

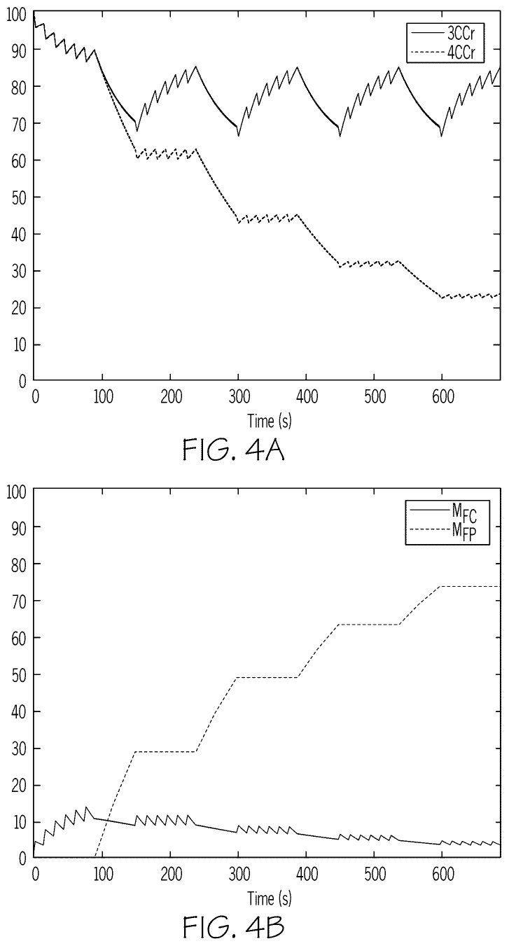

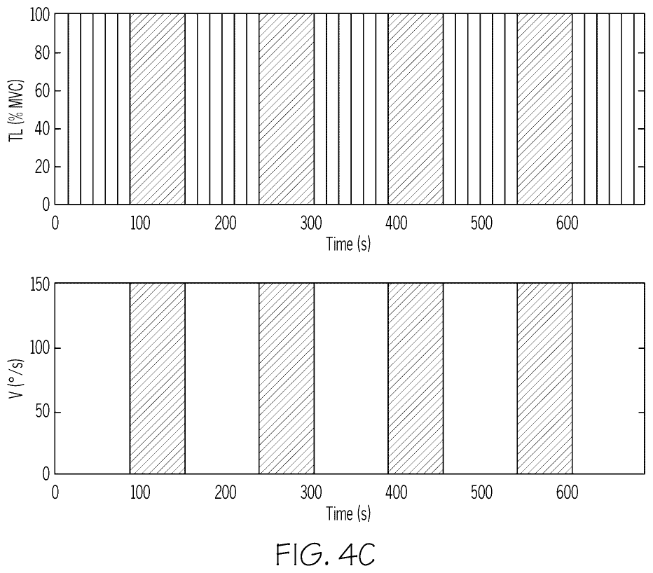

For Task 2, predicted residual capacities are identical during the first isometric bout as seen in FIG. 4A. This is to be expected since the 4CCr model is constructed to be equivalent to the 3CCr model in the trivial case. The predictions diverge from this point onwards. Due to a fraction of MUs moving into MFP which has a slow recovery rate, peripheral fatigue predicted by the 4CCr model is more persistent in the isokinetic phase than central fatigue predicted by the 3CCr model as seen in FIG. 4B, and RC therefore declines to a greater extent in the velocity-dependent 4CCr model. FIG. 4C depicts the variation of target load (top) and joint angular velocity (bottom) for Task 2.

For the next isometric phase, the 3CCr model allows the knee extensors to recover strength over the 90-second duration, whereas the 4CCr model maintains residual capacity (RC) at a nearly constant level as seen in FIG. 4A. For all subsequent isokinetic phases, decreases in RC are observed in both predictions, but are offset by recovery during the following isometric phases in the 3CCr model.

Allowed to run indefinitely, the 3CCr model predicts approximately 86% residual strength at the end of each isometric phase, excluding the first as seen in FIG. 4A. The 4CCr model, on the other hand, predicts decreasing strength losses in every isokinetic phase. Strength is seen to remain nearly constant or recover by a very small amount during the isometric phases. Since the extent of fatigue as predicted by the 3CCr model can be obtained simply as the complement of RC, the same conclusions can be drawn from both RC and muscle fatigue (MF) data. For the 4CCr model, however, two components of fatigue are simultaneously responsible and reveal different processes. Central fatigue rises, peaks, and then declines slowly as the activity progresses. Peripheral mechanisms, on the other hand, keep increasing the extent of fatigue after every isokinetic phase, as seen in FIG. 4B.

### Example 1.3. Discussion

The four-compartment controller model of LMF described here uses joint angular velocity along with target load to determine the relative contributions of central and peripheral mechanisms. Low velocities are dominated by central mechanisms, and increasing velocities allow peripheral mechanisms to assume a greater role. In its present formulation, all muscle properties are noted for an agonist muscle group, and the velocities referred to are the joint angular velocities resulting in concentric action (muscle shortening) of the agonists. If the same joint was articulated in the opposite direction, the agonists would then be lengthening against an increasing resistance while the shortening antagonistic group would be responsible for the vast majority of the motive force.

In this case, the TL for the agonistic group is considered 0 for simplicity, and that for the antagonistic group is non-zero. In reality, antagonistic muscles provide stability and fine motor control in eccentric contractions, so their contribution to the total torque output may not be exactly nil. A small enough contribution from antagonistic muscles, however, would not affect model predictions of RC for the agonists significantly as the model allows low intensity activities to continue almost indefinitely.

Experimental data on antagonistic force production in response to agonist contraction may be used to drive two separate simulations for the agonists and for the antagonists simultaneously to obtain more accurate predictions. Such an approach would be particularly well-suited to incorporation in a joint-based musculoskeletal model.

Being an extension of the 3CCr model, the new model reproduces its predictions exactly given the same task parameters and boundary conditions for isometric actions. This is important to ensure that in attempting to model more complex tasks with non-zero velocities, it does not lose its ability to accurately predict fatigue in zero-velocity tasks. It also ensures that the new model does not need to be revalidated for the trivial case as the 3CCr model has already been validated against extensive experimental data.

The 3CCr model was originally presumed to be exclusively representative of peripheral fatigue, but later studies have indicated that central processes may contribute much more to isometric tasks than peripheral ones alone. Additionally, the extremely rapid recovery observed in the predictions of the 3CCr model, especially during rest, is more characteristic of central mechanisms than peripheral ones, suggesting that it may in fact be predicting central fatigue at least for intermittent isometric tasks.

This does not detract from its general accuracy for the conditions it was validated against, and in borrowing from its general structure, the 4CCr model retains the same accuracy despite rebranding the only (peripherally) fatigued compartment as the centrally fatigued compartment in the new model. The 4CCr model's key contribution lies in the provision of a second fatigued state that allows a more nuanced consideration of the underlying fatigue/recovery phenomena while still requiring no measurement of biological signals. As more organized data from fundamental research into the dependence of fatigue mechanisms on tasks parameters is available, Equations 6-9 can easily be updated within the framework of this model to reflect the latest understanding of the physiology of fatigue.

### Example 1.4. Summary

In summary, Applicant proposes a mathematical four-compartment controller with augmented recovery (4CCr) model for predicting the change in maximal force production capacity of a muscle group over time. Based on compartment theory, this model can handle all activity types if the target load (TL) and joint velocity are both known as functions of time. It builds upon the established three-compartment controller with augmented recovery (3CCr) model which can accurately predict fatigue during isometric tasks, but whose predictions diverge significantly from experimental results during dynamic activity.

Under the 3CCr model, all motor units (MUs) within the muscle group (MG) are assumed to be in one of three possible states or compartments—active, resting, or fatigued. Active (M_A): Individual MUs generate force at their full capacity. The fraction of MUs in this state represents the MG's instantaneous force production as a fraction of its maximum force production capacity. Resting (M_R): Individual MUs produce no force but can be activated into force production rapidly using the neural drive. These represent the reserve capacity that can be called upon immediately. Fatigued (M_F): Individual MUs are incapable of force production and must move to the resting state before they can be activated. The neural drive is a proportional controller responsible for moving MUs between the active and resting states depending on the relative magnitudes of the TL, M_R and M_A, given by Equation 10.

$$C ( t ) = L \times min ( M\_R , TL - M\_A ) \qquad ( 10 )$$

The key innovation of the 4CCr model is replacing the fatigued compartment of the 3CCr model by two distinct compartments whose sizes depend on the relative contributions of both fatiguing mechanisms: Centrally fatigued (M_FC): MUs here have been fatigued by a central mechanism which dominates at zero and near-zero joint velocities. Recovery from this state is very rapid, of the order of a few seconds. Peripherally fatigued (M_FP): MUs here have been fatigued by a peripheral mechanism which dominates at higher velocities. Recovery from this state is slow, with full recovery taking hours. This new mathematical model can predict muscle fatigue for all human activity types.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A computer-implemented method of predicting muscle fatigue in a subject, wherein the method comprises:
receiving data related to muscle fatigue in a muscle of the subject;
feeding the data into an algorithm to predict the muscle fatigue, wherein the algorithm comprises at least the following models for predicting the muscle fatigue:
a) an active compartment (M_A) model representing individual motor units (MUs) generating force at full capacity,
b) a resting compartment (M_R) model representing inactive MUs capable of rapid activation into force production,
c) a centrally fatigued compartment (M_FC) model representing fatigued MUs due to a central mechanism dominant at zero or near-zero joint velocities, with rapid recovery, and
d) a peripherally fatigued compartment (M_FP) model representing fatigued MUs due to a peripheral mechanism dominant at higher velocities, with slow recovery; and
outputting the predicted muscle fatigue.

2. The method of claim 1, wherein the data related to muscle fatigue comprises joint velocity data.

3. The method of claim 1, wherein the data related to muscle fatigue comprises target load data.

4. The method of claim 1, wherein the data related to muscle fatigue comprises target load (TL) and joint velocity as functions of time for the muscle.

5. The method of claim 1, wherein the generation of force at full capacity of the M_A model is represented by MVC (maximum voluntary contraction).

6. The method of claim 1, wherein the M_A model is represented by the following formula:
$$\frac{{dM}_{A}}{dt} = - {F}_{P} {M}_{A} - {F}_{C} {M}_{A} + C ( t ) ,$$
wherein M_{A} represents the fraction of motor units activated,
wherein t represents time,
wherein F_{P} represents the peripheral fatigue coefficient,
wherein F_{C} represents the central fatigue coefficient, and
wherein C(t) represents the neural drive.

7. The method of claim 6, wherein C(t) is represented by the following formula:
$$C ( t ) = L \times min ( T L - {M}_{A} , {M}_{R} ) ,$$
wherein L represents a tracking factor,
wherein TL represents target load,
wherein M_{A} represents the fraction of motor units activated, and
wherein M_{R} represents the fraction of motor units at rest.

8. The method of claim 6, wherein F_{P} is represented by the following formula:
$${F}_{P} = {F}_{{P}_{0}} ( 1 - {e}^{- kV} ) ,$$
wherein F_{P0} represents the baseline peripheral fatigue coefficient,
wherein e represents the base of the natural logarithm function,
wherein k represents the velocity coefficient, and
wherein V represents the joint angular velocity.

9. The method of claim 6, wherein Fc is represented by the following formula:
$${F}_{C} = {F}_{{C}_{0}} {e}^{- kV} ,$$
wherein F_{C0} represents the baseline central fatigue coefficient,
wherein e represents the base of the natural logarithm function,
wherein k represents the velocity coefficient, and
wherein V represents the joint angular velocity.

10. The method of claim 1, wherein the M_R model is represented by the following formula:
$$\frac{{dM}_{R}}{dt} = {R}_{P} {M}_{FP} + {R}_{C} {M}_{FC} - C ( t ) ,$$
wherein M_{R} represents the fraction of motor units at rest,
wherein t represents time,
wherein R_{P} represents the peripheral fatigue coefficient,
wherein M_{FP} represents the fraction of motor units peripherally fatigued,
wherein R_{C} represents the central fatigue coefficient,
wherein M_{FC} represents the fraction of motor units, and
wherein C(t) represents the neural drive.

11. The method of claim 10, wherein C(t) is represented by the following formula:
$$C ( t ) = L \times min ( T L - {M}_{A} , {M}_{R} ) ,$$
wherein L represents a tracking factor,
wherein TL represents target load,
wherein M_{A} represents the fraction of motor units activated, and
wherein M_{R} represents the fraction of motor units at rest.

12. The method of claim 11, wherein R_{C} is represented by the following formula:
$${R}_{C} = \{ \begin{matrix}{R}_{{C}_{0}} if TL = 0 \\ {rR}_{{C}_{0}} if TL > 0\end{matrix} ,$$
wherein R_{C0} represents the baseline central fatigue coefficient,
wherein r represents the augmented recovery coefficient, and
wherein TL represents target load.

13. The method of claim 1, wherein the rapid recovery of the M_FC model is represented by a recovery time of less than about 1 hour.

14. The method of claim 1, wherein the zero or near-zero joint velocities of the M_FC model is represented by velocities ranging from zero to a positive value.

15. The method of claim 1, wherein the M_FC model is represented by the following formula:
$$\frac{{dM}_{FC}}{dt} = - {R}_{C} {M}_{F C} + {F}_{C} {M}_{A} ,$$
wherein M_{FC} represents the fraction of motor units centrally fatigued,
wherein t represents time,
wherein Rc represents central recovery coefficient,
wherein F_{c} represents the central fatigue coefficient, and
wherein M_{A} represents the fraction of motor units active.

16. The method of claim 1, wherein the peripheral mechanism of the M_FP model is represented by a rate of change of M_{FP}.

17. The method of claim 1, wherein the higher velocities of the M_FP model are represented by velocities above a positive value.

18. The method of claim 1, wherein the slow recovery of the M_FP model is represented by a recovery time of more than about 1 hour.

19. The method of claim 1, wherein the M_FP model is represented by the following formula:
$$\frac{{dM}_{FP}}{dt} = - {R}_{P} {M}_{FP} + {F}_{P} {M}_{A} ,$$
wherein M_{FP} represents the fraction of motor units peripherally fatigued,
wherein t represents time,
wherein R_{P} represents the peripheral recovery coefficient,
wherein F_{P} represents the peripheral fatigue coefficient, and
wherein M_{A} represents the fraction of motor units activated.

20. The method of claim 19, wherein R_{P} is represented by the following formula:
$${R}_{P} = {R}_{{P}_{0}} ,$$
wherein R_{P0} represents the baseline peripheral recovery coefficient.

21. The method of claim 1, wherein the outputting comprises generating a report of the predicted muscle fatigue.

22. The method of claim 1, wherein the outputting comprises displaying the predicted muscle fatigue on a screen.

23. The method of claim 1, wherein the muscle is selected from the group consisting of a skeletal muscle, a smooth muscle, or combinations thereof.

24. The method of claim 1, wherein the muscle comprises a skeletal muscle.

25. The method of claim 1, wherein the algorithm predicts muscle fatigue by predicting maximal force production capacity of the muscle over time.

26. The method of claim 1, wherein the predicted muscle fatigue comprises predicted muscle fatigue due to isometric muscle contraction.

27. The method of claim 1, wherein the predicted muscle fatigue comprises predicted muscle fatigue due to isokinetic muscle contraction.

28. The method of claim 27, wherein the predicted muscle fatigue due to isokinetic muscle contraction represents muscle fatigue due to dynamic tasks, repetitive tasks, or combinations thereof.

29. The method of claim 1, wherein the predicted muscle fatigue comprises predicted muscle fatigue due to isometric muscle contraction and isokinetic muscle contraction.

30. The method of claim 1, further comprising a step of recommending a treatment or minimization regimen for the muscle fatigue.

31. The method of claim 30, wherein the treatment or minimization regimen comprises modifications of tasks, modifications of workspaces, or combinations thereof.

32. The method of claim 30, wherein the treatment or minimization regimen is aimed at treating or minimizing a musculoskeletal disorder.

33. The method of claim 32, wherein the musculoskeletal disorder comprises trauma, back pain, arthritis, or combinations thereof.

34. The method of claim 1, further comprising a step of implementing a treatment or minimization regimen.

35. The method of claim 1, wherein the subject is a human being.

36. A computing device for predicting muscle fatigue in a subject, wherein the computing device comprises one or more computer readable storage mediums having a program code embodied therewith, wherein the program code comprises programming instructions for:
receiving data related to muscle fatigue in a muscle of the subject;
feeding the data into an algorithm to predict the muscle fatigue, wherein the algorithm comprises at least the following models for predicting the muscle fatigue:
a) an active compartment (M_A) model representing individual motor units (MUs) generating force at full capacity,
b) a resting compartment (M_R) model representing inactive MUs capable of rapid activation into force production,
c) a centrally fatigued compartment (M_FC) model representing fatigued MUs due to a central mechanism dominant at zero or near-zero joint velocities, with rapid recovery, and
d) a peripherally fatigued compartment (M_FP) model representing fatigued MUs due to a peripheral mechanism dominant at higher velocities, with slow recovery; and
outputting the predicted muscle fatigue.

37. The computing device of claim 36, wherein the program code further comprises programming instructions for recommending a treatment or minimization regimen for the muscle fatigue.
